(19) United States
(12) Patent Application Publication
(10) Pub. No.: US 2024/0307835 A1
(43) Pub. Date: Sep. 19, 2024

(54) APPARATUS FOR PREPARING A LIQUID PREPARATION

(71) Applicant: TRISTEL PLC, Newmarket (GB)

(72) Inventors: Jeremy TURNER, Newmarket (GB); Elanor DIXON, Newmarket (GB); James MORSE, Newmarket (GB); Thomas BRAND, Newmarket (GB)

(21) Appl. No.: 18/572,021

(22) PCT Filed: Jun. 23, 2022
(86) PCT No.: PCT/GB2022/051615

§ 371 Date: Dec. 19, 2023

(30) Foreign Application Priority Data
Jun. 23, 2021 (GB) 2108982.6

(51) Int. Cl. B01F 23/40; A61L 2/18; A61L 2/24; B01F 35/21; B01F 35/22; B01F 35/221; B01F 35/71; B01F 35/75; B01F 101/00; B01J 4/00; B01J 19/00; B01J 19/24

(52) U.S. Cl. CPC B01F 23/49; A61L 2/18; A61L 2/24; B01F 23/483; B01F 35/2111; B01F 35/2207; B01F 35/2211; B01F 35/22161; B01F 35/714; B01F 35/7547; B01J 4/008; B01J 19/0006; B01J 19/24; B01F 2101/2204; B01F 2101/2305; B01F 2215/045; B01J 2204/002; B01J 2219/00164

(57) ABSTRACT

An apparatus for preparing a liquid preparation uses a two-part system. A first part includes a first reagent and a second part includes a second reagent. The first reagent and the second reagent react when mixed to form an active ingredient of the liquid preparation. The liquid preparation is a diluent. The apparatus includes a primary chamber for receiving a quantity of the first part, a quantity of the second part and a quantity of the diluent, a reagent inlet for admitting the first part and the second part to the primary chamber, a diluent inlet connectable to a diluent supply for admitting the diluent to the primary chamber, an outlet for releasing the liquid preparation from the primary chamber, and a flow controller to admit a pre-determined volume of diluent to the primary chamber in a pre-determined time period when the diluent inlet is connected to a diluent supply.

### FIELD OF THE INVENTION

The present disclosure relates to an apparatus for preparing a liquid preparation, such as two-part disinfectant systems in which a first reagent and a second reagent react when mixed to form an active ingredient of the liquid preparation and a diluent is added to bring the preparation to a suitable concentration for use.

### BACKGROUND TO THE INVENTION

Many liquid preparations include active ingredients that degrade over time, limiting product shelf life. This is particularly true for disinfectants or sterilising agents such as chlorine dioxide, where the active ingredient is formed in situ when required by mixing two reagents. Examples are disclosed in WO 2005/011756. Chlorine dioxide, for example, may be formed by mixing a chlorite solution and an acid.

When preparing a liquid preparation of this type at the point of use, it is important to ensure that the resulting preparation has a suitable concentration of active ingredient. For a disinfectant composition, for example, an active ingredient concentration that is too low may result ineffective disinfecting activity, while an active ingredient concentration that is too high may result in a preparation that is unpleasant or hazardous in use. Accordingly, the user must follow a set of instructions to ensure that the reagents are added in the correct quantities, that the correct amount of diluent is used, and that sufficient time is allowed for the reaction to take place. Often, the instructions demand that the user takes several steps in a specific order, and one or more waiting periods may be mandated. Consequently, the preparation of such compositions can be time-consuming and labour-intensive, and potentially subject to human error.

It is known to provide pre-packaged quantities of the two parts of a two-part system that can be mixed together and with a suitable quantity of diluent, which is typically water. In one example, suitable quantities of the two parts are provided in separate sachets. The user adds an appropriate quantity of water to a container, then adds the contents of the two sachets, and waits for a prescribed time to allow the reaction to take place. Subsequently, a further quantity of water is added to bring the preparation to its working concentration, whereupon the preparation can be dispensed or decanted into another container for use. In such systems, the user must carefully observe instructions to ensure that sufficient reaction time has elapsed before the resulting preparation is used. In particular, it is not desirable to add the full volume of diluent to the container before adding the reagents, since the reaction rate would be inconveniently slow, so the diluent must be added in two separate steps. This also helps to ensure that the resulting solution is homogeneous.

In another example, WO 2017/060677 describes a dispensing capsule that has two or more sealed dispensing chambers each of which contains a different substance to be dispensed into a primary chamber. Screwing or pushing a cap onto the capsule causes progressive crushing of the walls of the dispensing chambers and breaks an internal seal between the chambers, permitting pre-mixing of the contents of the chambers to form a concentrated reagent mixture. Further crushing of the walls as the cap is screwed or pushed down results in breaking of an external seal, permitting discharge of the mixture into a primary chamber. The pre-mixing accelerates formation of the active agent. The reaction continues in the primary chamber, which contains a quantity of diluent, so that an adequate concentration of active agent is achieved in the primary chamber.

Advantageously, these arrangements allow the preparation of disinfectant preparations relatively quickly by using the correct quantity of concentrated reagents, without exposing the user to the concentrated reagents or to a concentrated reagent mixture. However, to ensure that the resulting disinfectant preparation has an effective concentration of active agent, the user must still adhere to a sequence of pre-determined process steps and timings. It is also important that the correct quantity of diluent is present in the primary chamber so that the resulting preparation has the correct concentration.

WO 2014/032832 discloses an apparatus having first and second reagent reservoirs that are connected to a mixing tank. The mixing tank is provided with a mains water inlet. Flow of the reagents and the water into the mixing tank is controlled by a valve system that is operable to admit the reagents and the water into the tank simultaneously. The valve system includes a float switch that shuts off the inflow of water when the contents of the mixing tank reach a predetermined volume and simultaneously opens an outlet of the mixing tank to drain the contents into another vessel. In this apparatus, the filling rate of the mixing tank, and hence the reaction rate of the diluted reagents, is dependent upon the mains water pressure and can therefore vary, providing inconsistent results. Furthermore, this apparatus requires a relatively complex valve system that increases costs and maintenance requirements.

Against that background, it would be desirable to provide methods and apparatus for the preparation of liquid preparations in which the user experience is simplified, and/or in which the risk of human error is reduced.

### SUMMARY OF THE INVENTION

Aspects of the invention are specified in the independent claims. Preferred features are specified in the dependent claims.

In a preferred embodiment, the invention provides apparatus for preparing a liquid preparation using a two-part system comprising a first part including a first reagent and a second part including a second reagent, in which the first reagent and the second reagent react when mixed to form an active ingredient of the liquid preparation, and in which the liquid preparation comprises a diluent. The apparatus comprises a primary chamber for receiving a quantity of the first part, a quantity of the second part and a quantity of the diluent, a reagent inlet for admitting the first part and the second part to the primary chamber, a diluent inlet connectable to a diluent supply for admitting the diluent to the primary chamber, an outlet for releasing the liquid preparation from the primary chamber and a flow controller configured to admit a pre-determined volume of diluent to the primary chamber in a pre-determined time period when the diluent inlet is connected to a diluent supply.

With this arrangement, the correct volume of diluent can be added to the primary chamber by monitoring only the filling time of the diluent. In this way, the user need not continuously monitor the filling process, nor make volumetric measurements of the diluent, allowing for a simplified preparation process that is less susceptible to error. Furthermore, the pre-determined time period over which the pre-determined volume of diluent is admitted can be selected to optimise the reaction between the first and second reagents, for example by avoiding premature dilution of the reagents.

The flow controller may comprise a flow restrictor configured to allow diluent to flow into the primary chamber at a pre-determined inlet flow rate, provided the diluent supply has an unrestricted flow rate greater than the pre-determined inlet flow rate. Preferably, the pre-determined inlet flow rate is between 2.5 and 10 litres per minute. In an example, the primary chamber has a capacity of at least 10 L, the pre-determined flow rate is 4.9 litres per minute, and the quantities of the first and second parts have a combined volume of 200 mL. In this example, a total filling time of two minutes is required to produce a final volume of 10 L of the liquid preparation.

The first and second parts may be supplied in packages, such as sachets, that contain a suitable quantity of each of the parts. The packages may be opened and emptied into the reagent inlet manually by the user. Alternatively, the apparatus may include features to automate or assist the process of adding the first and second parts to the primary chamber.

For example, the apparatus may comprise a receiving region for receiving a capsule having first and second cavities for storing the respective first and second parts, and may include an actuator operable to cause dispensing of the first and second parts from the capsule into the reagent inlet. The actuator may, for example, be part of a closable lid for the apparatus.

In an embodiment, the reagent inlet comprises a reaction chamber for receiving the first and second parts, the reaction chamber having an outlet for delivering a reagent mixture formed from the first and second parts to the primary chamber. The outlet may comprise a restriction orifice for releasing the reagent mixture into the primary chamber at a controlled rate, and/or a valve for controlling the release of the reagent mixture from the reaction chamber. By allowing the reagents to dwell in a reaction chamber before dilution, the reaction proceeds rapidly and the overall preparation time is reduced.

The flow controller preferably comprises a fill valve operable to stop and start the flow of diluent into the primary chamber. The fill valve may be manually operable, in which case the user is responsible for operating the valve for the pre-determined time period, as may be prescribed by operating instructions. Alternatively, the fill valve may be operable by the apparatus. For example, the fill valve may be a solenoid-controlled valve.

The apparatus may comprise a control module configured to receive a reagent delivery signal associated with the delivery of the first part and the second part to the primary chamber, and to cause operation of the fill valve in response to the reagent delivery signal. The control module may be configured to provide a ready indication to the user after admission of the pre-determined quantity of diluent to the primary chamber to indicate that the preparation is ready for use. The ready indication may be an audible and/or visual indication provided by an indicator device, such as one or more indicator lights, a display screen and/or a loudspeaker.

The control module may be configured to wait for a pre-determined dwell period after admission of the pre-determined quantity of diluent to the primary chamber before providing the ready indication, so that sufficient time is provided for the reaction to occur. Similarly, the control module may be configured to wait for a pre-determined reaction period after receiving the reagent delivery signal before admitting the pre-determined quantity of diluent to the primary chamber, to allow the reagents to react before dilution.

In some embodiments, the pre-determined quantity of diluent is added to the primary chamber in two charges, before and after the addition of the first and second parts. Thus the control module may be configured to receive a start input signal before delivery of the first part and the second part to the primary chamber, and to cause operation of the fill valve in response to the start input signal to deliver a pre-delivery charge of the diluent to the primary chamber. Preferably, operation of the fill valve in response to the reagent delivery signal causes delivery of a post-delivery charge of the diluent to the primary chamber, the pre-delivery charge and post-delivery charges together having a total volume equal to said pre-determined quantity of diluent. The control module may be configured to provide a fill ready indication to the user after delivery of the pre-delivery charge to indicate that the first part and the second part can be added to the primary chamber.

In other embodiments, the pre-determined quantity of diluent is added to the primary chamber in a single charge. In such cases, operation of the fill valve in response to the reagent delivery signal admits the pre-determined volume of diluent to the primary chamber, and the control module may be configured to provide a fill ready indication to the user to indicate that the first part and the second part can be added to the primary chamber before, after or during delivery of the diluent.

The apparatus may include a user input device operable by a user to cause the user interface to provide the reagent delivery signal to the control module. When the control module is also configured to receive a start input signal, the user input device may be operable by the user to cause the user interface to provide the start input signal to the control module. The user interface may, for example, include buttons or switches for operation by the user, or may be a touchscreen that also acts as an indicator device.

The apparatus may be configured to provide the reagent delivery signal without direct user input. For example, the apparatus may comprise a delivery sensor configured to sense an event associated with the delivery of the first part and the second part to the primary chamber and to provide the reagent delivery signal to the control module upon sensing of the event. When the apparatus includes a receiving region for receiving a capsule and an actuator operable to cause dispensing of the first and second parts from the capsule, the delivery sensor may be configured to sense operation of the actuator and to provide the reagent delivery signal to the control module upon sensing of said operation.

The apparatus may include a diluent inlet flow sensor, and the control module may be configured to receive an inlet flow signal from the inlet flow sensor and to determine a diluent inlet flow rate based on the inlet flow signal. The control module may be configured to adjust an opening time of the fill valve based on the inlet flow rate. Alternatively, or in addition, the control module may be configured to provide a fault indication to the user if the inlet flow rate is outside a pre-determined range.

The apparatus may include a fault sensor for detecting the presence of liquid in the primary chamber. The control module may be configured to receive a fault signal from the fault sensor and to provide a fault indication to the user if the presence of liquid in the primary chamber is detected before operation of the fill valve. The fault sensor may, for example, comprise a back pressure sensor for detecting a back pressure of liquid in the primary chamber at the diluent inlet.

The apparatus may include a data reader, such as an RFID reader, for reading data from a data carrier on a package containing the first part and/or the second part. The data may relate to properties of the first part and/or the second part, such as chemistry type, batch number, expiry date, shelf life and so on. The control module may be configured to receive the data from the data reader, validate the data against one or more pre-determined criteria and, if the data does not satisfy the one or more pre-determined criteria, provide an error indication to the user prior to operation of the fill valve.

The apparatus may comprise a drain port for emptying the primary chamber. The drain port may have a drain valve operable by the control module, so that the contents of the primary chamber can be automatically emptied to drain. For example, the control module may be configured to operate the drain valve to empty the primary chamber once a pre-determined storage life of the liquid preparation has been exceeded.

An outlet flow sensor may be provided for sensing flow of the liquid preparation through the outlet. The control module may be configured to determine, from an output of the outlet flow sensor, an outlet flow volume when the liquid preparation is released from the outlet, and to log said outlet flow volume. The logged flow volume data can be used for stock control, auditing, usage analysis and so on.

The outlet may include an outlet valve, which may be operable by the control module to control flow through the outlet. In this way, the control module can restrict flow through the outlet when necessary. For instance, the control module may be configured to operate the outlet valve to prevent flow through the outlet during preparation of the liquid preparation.

Preferred and/or optional features of each aspect and embodiment of the invention may also be used, alone or in appropriate combination, in the other aspects and embodiments also.

### BRIEF DESCRIPTION OF THE DRAWINGS

Embodiments of the present invention will now be described, by way of example only, with reference to the accompanying drawings in which like reference signs are used for like features, and in which:

FIG. 1 is a schematic illustration of an apparatus according to a first embodiment of the invention;

FIG. 2 shows a sequence of steps in a method of operating the apparatus of FIG. 1;

FIG. 3 is a schematic illustration of an apparatus according to a second embodiment of the invention;

FIG. 4 shows a sequence of steps in a method of operating the apparatus of FIG. 3; and

FIGS. 5 to 10 are schematic illustrations of apparatus according to further embodiments of the invention.

### DESCRIPTION OF THE PREFERRED EMBODIMENTS

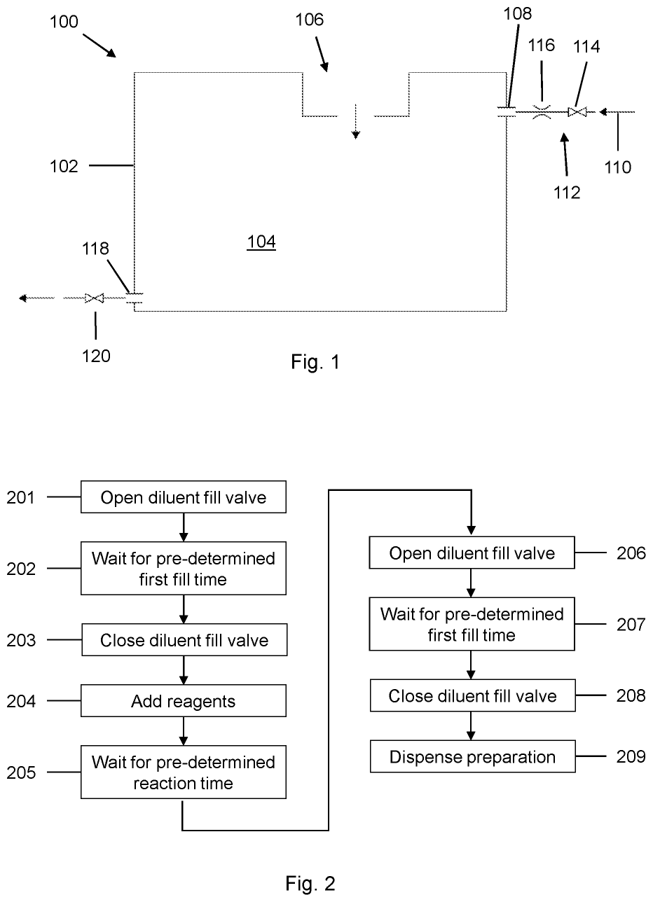

An apparatus 100 according to a first embodiment of the invention is shown in FIG. 1. The apparatus 100 comprises a tank 102 that encloses a primary chamber 104. A reagent inlet 106 is disposed in the top of the tank 102 and allows reagents to be added to the primary chamber 104.

A diluent inlet 108 is provided to admit a diluent liquid, such as water, to the primary chamber 104. The diluent inlet 108 can be connected to a diluent supply (indicated by arrow 110) capable of providing a continuous supply of diluent. For example, the diluent supply 110 may be a mains water supply line. Flow of diluent from the diluent supply 110 through the diluent inlet 108 into the primary chamber 104 is controlled by a flow controller 112. The flow controller 112 includes a fill valve 114 and a flow restrictor 116. In this embodiment, the fill valve 114 can be operated by a user (for example by turning a handle, or by solenoid control) to start or stop the flow of diluent. The flow restrictor 116 limits the flow rate of diluent into the primary chamber 104.

The apparatus 100 also includes an outlet 118 for releasing the contents of the primary chamber 104 for use after the liquid preparation has been successfully prepared. Flow through the outlet 118 is controlled by an outlet valve 120 that is operable by the user.

The apparatus 100 is intended for preparing a liquid preparation using a two-part system comprising a first part including a first reagent and a second part including a second reagent. The first reagent and the second reagent react when mixed to form an active ingredient of the liquid preparation, and the diluent serves to dilute the active ingredient to an appropriate efficacious concentration. At least one, and preferably both, of the first and second parts are of liquid form.

The reagents used in the system may, for example, be reagents which when mixed produce a disinfectant composition; for example chlorine dioxide or peracetic acid. Suitable reagents will be well known to those skilled in the art; for example, reagents for producing chlorine dioxide include: chlorite and acid; chlorate, peroxide and acid; and chlorite, hypochlorite, and a suitable buffer. The reagents may be in a concentrated form, providing rapid formation of the active agent when the first and second parts are mixed.

Advantageously, in the apparatus 100 of FIG. 1, the flow restrictor 116 is selected so that diluent will flow into the primary chamber 104 at a known, pre-determined rate when the fill valve 114 is open, provided that the diluent supply 110 is capable of supplying the diluent at a higher flow rate than that permitted by the flow restrictor 116. This substantially simplifies the process of charging the primary chamber 104 with a desired amount of diluent, since a known amount of diluent will flow in a given time. In this way, the user can perform a diluent fill step by opening the fill valve 114, allowing a pre-determined length of time to pass, and then closing the fill valve. It is not necessary for the user routinely to make a physical measurement of the volume of liquid nor to continuously monitor the apparatus during the filling time.

The flow restrictor 116 may restrict liquid flow using a suitably-sized restricted orifice, although any suitable type of flow restrictor 116 can be used.

In one example, the diluent supply 110 is a mains water supply, the nominal capacity of the primary chamber is 10 L, and 100 mL of each of the first and second parts of the two-part system are provided in separate sachets. In this case, the flow restrictor 116 preferably limits the flow rate to a maximum of 4.9 litres per minute. This is below the usual flow rate available from a mains water supply (in the UK, for example, unrestricted mains water flow rates of between 10 and 15 litres per minute are typical). Accordingly, it can be expected that, in normal use, water will flow into the primary chamber 104 at a rate of 4.9 litres per minute due to the flow restrictor 116.

Referring additionally to FIG. 2, the apparatus 100 can be used to prepare a disinfectant composition as follows.

First, in step 201, the user opens the fill valve 114. This causes water to flow from the supply line 110 into the primary chamber 104 through the diluent inlet 108 at the rate determined by the flow restrictor 116. In step 202, the user waits for a pre-determined time period and then, once the time period has elapsed, in step 203 the user closes the fill valve 114. In this way, a first or pre-delivery charge of diluent is added into the primary chamber 104. The volume of the pre-delivery charge is the product of the flow rate of the flow restrictor 116 and the pre-determined time period in step 202. For the example flow rate of 4.9 litres per minute, and a time period of one minute, the pre-delivery charge will be 4.9 L.

Next, in step 204, the reagents are added to the primary chamber 104. Thus the user opens the supplied sachets containing the pre-determined quantities of the first and second parts and pours the contents into the primary chamber 104 through the reagent inlet 106. In step 205, the user waits for a pre-determined reaction time period. The reaction time period will be prescribed in the instructions, and will depend upon the reaction rate and the concentrations of the first and second parts. It is conceivable that no waiting time will be required in step 205.

After the reagents have been added and any necessary reaction time has elapsed, a second or post-delivery charge of diluent is added into the primary chamber 104. Thus the user opens the diluent fill valve 114 in step 206, waits for a pre-determined time period in step 207, and closes the diluent fill valve 114 in step 208. Again, the volume of this post-delivery charge is the product of the flow rate of the flow restrictor 116 and the pre-determined time period in step 207, and for the example flow rate of 4.9 litres per minute, and a time period of one minute, the post-delivery charge will be 4.9 L.

A total of diluent quantity of 9.8 L of diluent is therefore added in steps 201 to 208, which combines with 100 mL of the first part and 100 mL of the second part to provide a total of 10 L of the liquid preparation in the primary chamber 104 at the end of step 208. Subsequently, in step 209, the user can dispense a desired volume of the liquid preparation through the outlet 118 by operating the outlet valve 120.

It will be appreciated that, in its simplest form, the apparatus 100 may be purely mechanical in nature with manual control of the fill valve 114 and the outlet valve 120, and the user may use an external clock to time the pre-determined time periods in accordance with supplied instructions. However, it will be understood that, in variants, the apparatus 100 may include an integrated timer that may be pre-programmed with the appropriate time intervals, and may also prompt the user to take the required actions at the appropriate times. It is also conceivable that the fill valve 114 could be solenoid-operated and controlled by the timer, so that the user can open the fill valve 114 for the desired time period by activating a start control of the timer.

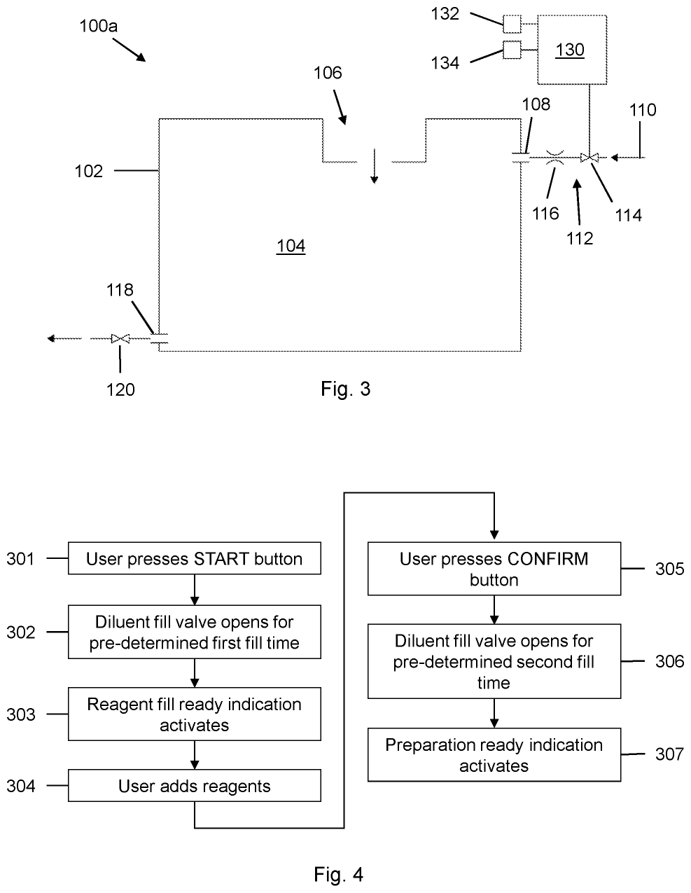

FIG. 3 shows apparatus 100a according to a second embodiment of the invention which is similar to the first embodiment (and only the differences will be described). In this second embodiment, the fill valve 114 is operated by a control module 130. The control module 130 is connected to a user interface 132 for supplying user commands to the control module 130, and to an indicator device 134 for providing indications to the user. The indicator device 134 may comprise one or more lights, a display screen, an audio output or any combination of these, and may be combined or integrated with the user interface 132, for example in a touchscreen. The control module 130 is preferably microprocessor-based, although it would also be possible to implement the control module 130 through other means. The control module 130 is pre-programmed with the appropriate sequence of steps and pre-determined fill and wait times for the correct preparation of the desired composition.

Referring also to FIG. 4, to prepare a liquid preparation using the apparatus 100a of FIG. 3, in step 301, the user first checks that the apparatus 100a is free of damage and that the outlet valve 120 is closed, then presses a “start” button on the user interface 132. This causes a start input signal to be transmitted to the control module 130. On receiving the start input signal, in step 302 the control module 130 causes the fill valve 114 to open for the pre-determined first fill time, and then to close again. This charges the primary chamber 104 with the correct volume of diluent for the pre-delivery charge, with the volume of the pre-delivery charge being determined by the flow rate of the flow restrictor 116 and the length of the first fill time.

Then, at step 303, the control module 130 causes the indicator device 134 to provide an audio and/or visual “reagent fill ready” indication to the user, to prompt the user to add the reagents to the primary chamber 104 through the reagent inlet 106, as described above. In step 304, prompted by the indication, the user adds the reagents as described above.

Then, in step 305, the user presses a “confirm” button on the user interface 132. This causes a reagent delivery signal to be received by the control module 130. After receiving the reagent delivery signal, in step 306 the control module 130 causes the fill valve 114 to open for the pre-determined second fill time, and then to close again, to charge the primary chamber 104 with the correct volume of diluent for the post-delivery charge. If required, the control module 130 may wait for a pre-determined reaction time to elapse after receiving the reagent delivery signal before causing the fill valve 114 to open in step 306. Finally, in step 307, the control module 130 causes a “preparation ready” indication to be provided to the user through the indicator device 134.

It will be understood that the user interface 132 and control module 130 could be configured so that a single operating button could be pressed both to trigger the start of the process in step 301 and to confirm that the reagent has been added in step 305.

In the embodiments of FIGS. 1 to 4, the flow restrictor 116 is used to ensure that a pre-determined volume of diluent is admitted to the primary chamber in a pre-determined time period, provided that the unrestricted flow rate of the diluent supply 110 is greater than the flow rate of the flow restrictor 116. The tank 102 may be designed so that the liquid level in the primary chamber 104 can be visually checked, such as by making all or part of the tank from a transparent material, and providing level markings corresponding to the liquid levels expected after the pre-delivery and post-delivery charges respectively. Alternatively, or in addition, a level gauge and/or an electronic level sensor could be provided. In these ways, the user can identify incorrect filling due to the flow rate of the diluent supply 110 dropping below the flow rate of the flow restrictor 116 or due to component failure or other errors.

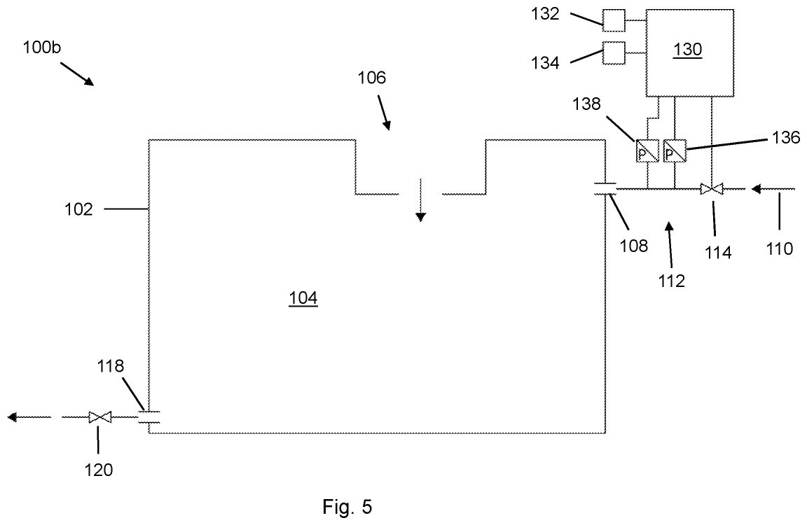

FIG. 5 shows apparatus 100b according to a third embodiment of the invention, which is similar to the second embodiment (and only the differences will be described). In this third embodiment, the flow restrictor 116 is omitted, and the flow controller 112 comprises only the fill valve 114 which is operable by the control module 130 to stop and start the flow of diluent into the primary chamber 102.

The apparatus 100b includes a diluent inlet flow sensor, which in this example is an inlet pressure sensor 136 arranged to sense the pressure of the inflowing diluent between the fill valve 114 and the diluent inlet 108 when the fill valve 114 is open. The control module 130 receives an inlet pressure signal from the inlet pressure sensor 136 and can determine a diluent inlet flow rate based on the inlet pressure signal. The control module 130 can then determine and dynamically adjust the opening times of the fill valve 114 based on the inlet flow rate so that the desired quantity of diluent is admitted to the primary chamber 104. The control module 130 is also configured to provide a fault indication to the user through the indicator device 134 if the inlet pressure is outside a pre-determined range. In an example, the pre-determined pressure range corresponds to an acceptable flow rate range of between 2.5 and 10 litres per minute, although flow rates outside of this range may also be acceptable depending on the size of the primary chamber 104 and the chemistry type.

In this embodiment, the apparatus 100b also includes a back pressure sensor 138, arranged to detect a back pressure between the diluent inlet 108 and the fill valve 114. The back pressure sensor 138 provides a fault sensor that can detect the presence of liquid in the primary chamber 104 and provides a fault signal to the control module 130. The control module 130 is configured to provide a fault indication to the user through the indicator device 134 if the presence of liquid in the primary chamber 104 is detected before operation of the fill valve 114, which would indicate that the primary chamber 104 has not been emptied before starting a preparation process.

The process of preparing a liquid preparation using the apparatus 100b of FIG. 5 is generally similar to that for the apparatus 100a of FIG. 3. However, during the first and second fill times, the control module 130 monitors the water flow, using the inlet pressure sensor 136, and calculates the end of the fill time so that the fill valve 114 shuts off once the appropriate volume of diluent has been added. Additionally, during the first and second fill times, the inlet pressure sensor 136 and back pressure sensor 138 are monitored by the control module 130 for signals that indicate an out-of-range inlet flow or an unexpected back pressure, as described above.

The inlet pressure sensor 136 and back pressure sensor 138 may be provided by a single sensor unit, or by separate sensors. Instead of an inlet pressure sensor, any suitable type of flow sensor may be provided to allow determination of the inlet flow rate, which may be measured directly (such as by a turbine sensor) or indirectly (as is the case for a pressure sensor). Similarly, instead of a back pressure sensor 138, any suitable type of fault sensor capable of detecting liquid in the primary chamber 104 may be provided. Examples include resistance-based liquid sensors and float-switch based sensors. The fault sensor may be installed at or close to the base of the primary chamber 104.

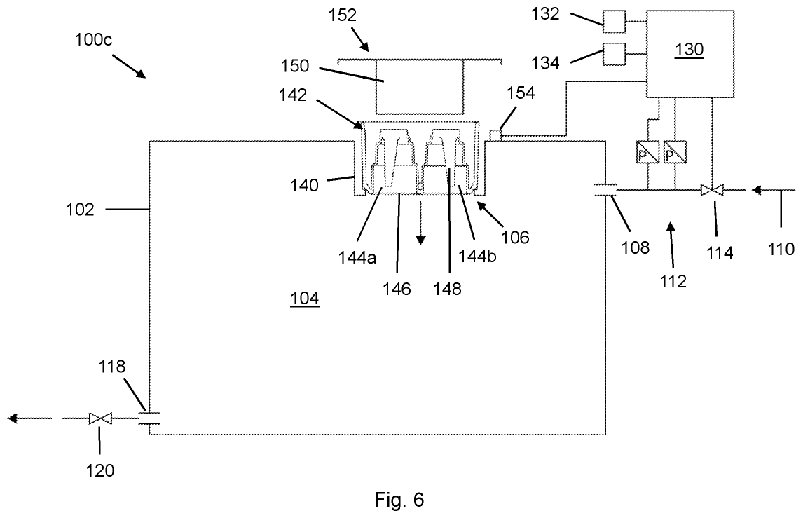

FIG. 6 shows apparatus 100c according to a fourth embodiment of the invention, which is similar to the third embodiment (and only the differences will be described).

In this embodiment, the tank 102 includes a receiving region 140 for receiving a capsule 142 having first and second cavities 144a, 144b for storing the respective first and second parts, for example as described in WO 2017/060677, the contents of which are incorporated herein by reference. The cavities 144a, 144b are defined by collapsible walls, and are sealed at their lower ends by a foil seal 146. Each cavity includes a burst pin 148 that extends downwardly towards the seal 146.

The apparatus 100c also includes an actuator, in the form of a piston 150, that is operable to cause dispensing of the first and second parts from the capsule 142 into the reagent inlet 106. The piston 150 is mounted to a lid 152 that can be lifted to allow access to the receiving region 140, so that the capsule 142 can be inserted, and closed so that the piston 150 applies pressure to the walls that define the cavities 144a, 144b so that the walls begin to collapse. The resulting increase in pressure in the cavities 144a, 144b causes the seal 146 initially to detach from a dividing member that separates the cavities 144a, 144b, while remaining attached to a periphery of the capsule 142. This allows the first and second parts to mix and react, to an extent, while still contained within the capsule. Subsequently, as the piston 150 is pushed further onto the capsule 142, the burst pins 148 contact the seal 146 and burst the seal 146 or detach it from at least a part of the periphery, to allow the mixture of the first and second parts to flow into the primary chamber 104 through the reagent inlet 106. The lid 152 may be connected to the tank 102 at a hinge (not shown), so that the lid 152 can be conveniently opened and closed while still attached to the tank 102.

The apparatus 100c is provided with a reagent delivery sensor 154 that is configured to sense operation of the piston 150 and to provide a reagent delivery signal to the control module 130. In this example, the reagent delivery sensor 154 is in the form of a microswitch arranged to be activated when the lid is closed. In this way, the control module 130 can determine when the lid 152 has been closed, triggering the release of the contents of the capsule 142. When the lid 152 is opened, the reagent delivery sensor 154 returns to a reset state, so that the control module 130 can determine when a new capsule 142 has been inserted.

In this embodiment, the required amount of diluent is added to the primary chamber 104 in a single charge, after the reagent has been delivered to the primary chamber 104. Therefore, to prepare the preparation, the user first ensures that the outlet valve 120 is closed, and then loads a capsule 142 into the receiving region 140. The user then closes the lid 152 to activate the bursting mechanism, forcing the piston 150 onto the capsule 142 and dispensing its contents into the primary chamber 104 as described above. This action causes the reagent delivery sensor 154 to provide the reagent delivery signal to the control module 130, which then opens the fill valve 114 to start the diluent fill. As in the previous embodiment, the control module 130 monitors the inlet flow rate and shuts off the fill valve 114 once the required volume of diluent has been added.

It will be appreciated that additional or alternative types of reagent delivery sensor 154 could be provided. For example, an optical sensor may be used to detect the flow of liquid from the capsule 142. One or more additional sensors may be used for fault detection or for initiating the control module 130 at the start of an operation. For instance, a microswitch or proximity detector could be provided to detect the presence of a capsule 142 in the receiving region 140.

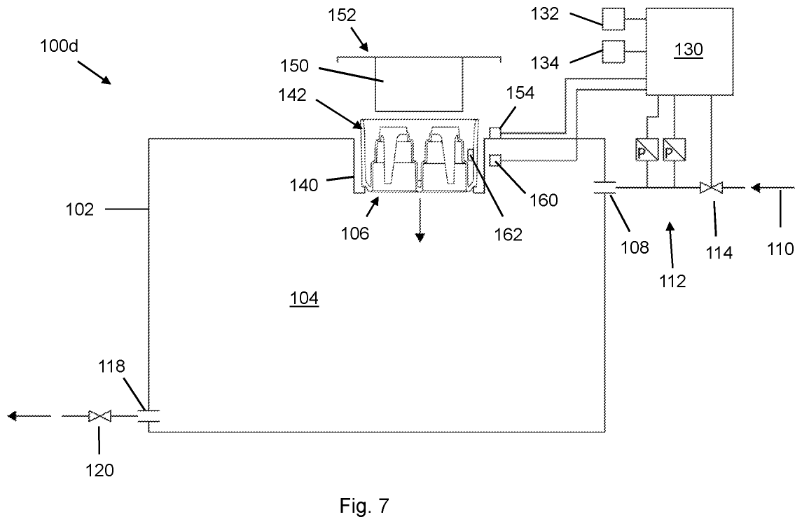

FIG. 7 shows apparatus 100d according to a fifth embodiment of the invention, which is similar to the fourth embodiment, but with the addition of an RFID data reader 160 for reading data from a data carrier comprising an RFID tag 162 provided on the capsule 142 when the capsule 142 is in place in the receiving region 140.

The data encoded on the tag 162 provides information relating to the properties of the reagents contained within the capsule 142. For example, the data may include the type of chemistry (e.g. the composition and concentration of the reagents), a batch number, an expiration date of the capsule (based on the expected shelf life of the reagents before mixing), and a shelf life of the resulting liquid preparation, which can be used to calculate an expiry time of the liquid preparation based on the time and date at which the preparation is made.

The control module 130 is configured to receive the data from the data reader 160 and validate the data against one or more pre-determined criteria. If the data does not satisfy the one or more pre-determined criteria, the control module 130 provides an error indication to the user through the indicator device 134.

In use, when the capsule 142 is loaded into the receiving region 140, the data on the tag 162 is read, and the control module 130 checks that the capsule 142 is in date, and may provide confirmation of this, and the capsule chemistry type, using the indicator device 134 (which in this embodiment includes a display). Once the diluent has been added and the liquid preparation is ready for use, the control module 130 causes the indicator device 134 to display a countdown indicating the remaining shelf life of the preparation, together with the chemistry type and the lot number for logging and validation purposes. In the event that the capsule 142 is already beyond its expiry date, the control module 130 causes an error indication to be provided.

The data provided on the tag 162 may also include details of dwell times and diluent fill volumes required for correct preparation. These details can be used by the control module 130 to set appropriate wait times and charge volumes for the various steps in the preparation sequence. In this way, the apparatus 100d can be automatically set up for use with different types of chemistry, for example to produce cleaning solutions, disinfecting solutions and rinsing solutions in the same unit.

The control module 130 may use the received data to detect that a capsule 142 with a different chemistry type to that used in the previous process has been inserted and, if appropriate, prompt and assist the user in performing a water flush of the primary chamber 104.

While RFID provides a convenient way to transfer data from the capsule 142 to the apparatus 100d, different machine-readable data formats, such as bar codes or matrix codes, are also suitable. It will also be understood that the use of a data reader in this way is not limited to embodiments in which a capsule 142 is provided, and it would be equally possible to provide suitable data carriers on reagent packages such as sachets or bottles, and to provide a data reader in a convenient location on the apparatus to allow the data to be read from the packages before use.

The control module 130 may also count the number of cycles that have been performed, and prompt the user to carry out a flush of the primary chamber 104 and/or a descaling operation.

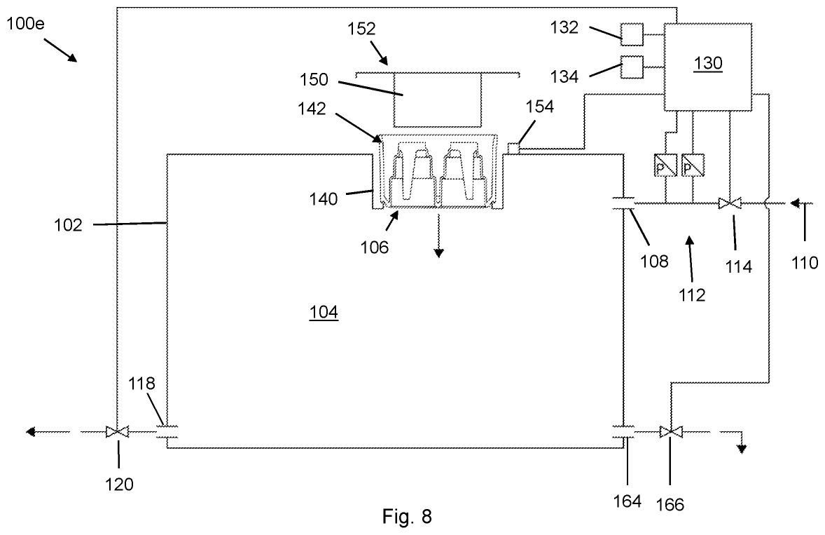

FIG. 8 shows apparatus 100e according to a sixth embodiment of the invention, which is similar to the fourth embodiment described above with reference to FIG. 6.

In this sixth embodiment, the tank 102 is provided with a drain port 164 that allows the contents of the primary chamber 104 to be drained. The drain port 164 can be connected to mains drainage or to a waste container as appropriate. Flow through the drain port 164 is controlled by a drain valve 166 that is operated by the control module 130.

The control module 130 can be configured so that, when the shelf life of the preparation has expired, the drain valve 166 is opened automatically to drain any remaining liquid from the primary chamber 104. In this way, there is no opportunity for a user to inadvertently use the preparation when it is too old to be effective.

Additionally, in this embodiment, the outlet valve 120 is also operated by the control module 130. This allows the control module 130 to ensure that the outlet valve 120 is closed before the fill valve 114 is opened, to reduce the risk of spillage or flood, and to ensure also that the outlet valve 120 remains closed during filling and dwell periods before the preparation is ready.

The user may dispense the liquid preparation using a suitable button or other control to instruct the control module 130 to open the outlet valve 120, or by manually overriding the outlet valve 120. Advantageously, if the flow rate through the outlet 118 is constant, this allows the outlet valve 120 to act as a flow sensor. Accordingly, the control module 130 can set an opening time for the outlet valve 120 so that a pre-determined quantity of preparation can be dispensed, or can measure the time that the outlet valve 120 is open if the opening time is controlled by the user or if the valve 120 is manually overridden, to allow the amount of preparation dispensed to be determined and logged. A dedicated flow sensor or flow controller (not shown) may also be used to provide this functionality. The data obtained in these ways can be analysed to establish the volume of preparation that is required for cleaning or disinfection in a particular environment, for stock management (such as automatically ordering or prompting the order of new capsules or other reagent packages), and for auditing purposes (where, for example, a change in cleaning practices could be detected by a change in the amount of preparation being used).

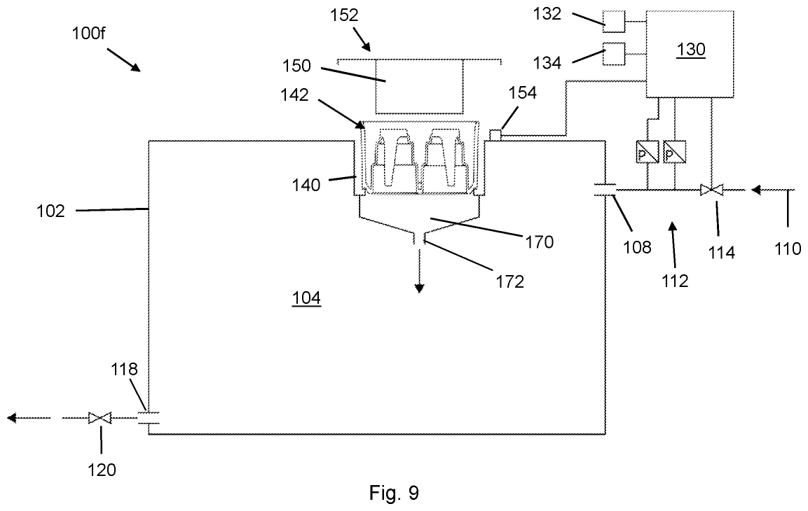

FIG. 9 shows apparatus 100f according to a seventh embodiment of the invention, which is similar to the fourth embodiment described above with reference to FIG. 6 (and only the differences will be described). In this embodiment, the reagent inlet 106 includes a reaction chamber 170 disposed below the receiving region 140 so that, when the first and second parts are released from the capsule 142, they are received in the reaction chamber 170. The reaction chamber 170 has an outlet in the form of a restricted orifice 172, through which the contents of the reaction chamber 170 can flow at a controlled rate into the primary chamber 104. With this arrangement, the reagents can mix and react in the reaction chamber 170 before being diluted in the primary chamber 104. This means that the preparation time can be reduced, since the reaction time of the concentrated reagents is reduced, and diluent filling can take place simultaneously without a negative impact on the reaction time.

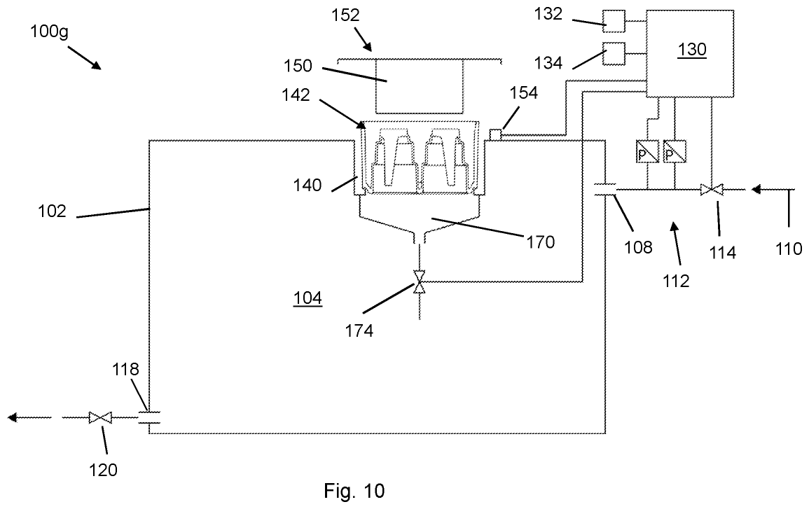

FIG. 10 shows apparatus 100g according to an eighth embodiment of the invention, which is a variant of the seventh embodiment shown in FIG. 9. In this case, flow through the outlet of the reaction chamber 170 is controlled by a dispensing valve 174 that is operable by the control module 130. With this arrangement, the dwell time of the concentrated reagents in the reaction chamber 170 can be precisely controlled by the control module 130, so that the preparation time can be optimised and adjusted for different chemistry types.

It will be appreciated that the features presented above in different embodiments may be used in combinations other than those shown. For example, the reaction chamber arrangements of FIGS. 9 and 10 could be used in any of the other embodiments of the invention. The capsule arrangements shown in FIGS. 6 to 10 could equally be used in the arrangements of FIGS. 1 and 3 and, conversely, the capsule arrangements in FIGS. 6 to 10 could be omitted in favour of use with sachets, bottles or other packages. More generally, the skilled person will understand that the various features disclosed in each embodiment could be used in any suitable combination.

Various further modifications can be contemplated. For example, the diluent inlet may be fitted with a flow restrictor with a variable flow rate that can be controlled by the control module, to allow faster or slower filling when appropriate and/or to compensate for differences in the diluent source supply pressure. Automated systems, under the control of the control module, could be provided for automatically dispensing the contents of the capsule or other reagent packages at an appropriate time. It would also be possible to store larger quantities of each reagent in separate reservoirs and use dispensing valves, under the control of the control module, to dispense aliquots of each reagent into the primary chamber or a reaction chamber when required. It is also conceivable that the apparatus could be used to prepare a liquid preparation using a single-part system, in which an active ingredient is added directly into the reagent inlet for subsequent dilution in the primary chamber.

Further modifications and variations not explicitly described above can also be contemplated without departing from the scope of the invention as defined in the appended claims.

1. An apparatus for preparing a liquid preparation using a two-part system comprising a first part including a first reagent and a second part including a second reagent, wherein the first reagent and the second reagent react when mixed to form an active ingredient of the liquid preparation, and wherein the liquid preparation comprises a diluent;
the apparatus comprising:
a primary chamber for receiving a quantity of the first part, a quantity of the second part and a quantity of the diluent;
a reagent inlet for admitting the first part and the second part to the primary chamber;
a diluent inlet connectable to a diluent supply for admitting the diluent to the primary chamber;
an outlet for releasing the liquid preparation from the primary chamber;
and a flow controller configured to admit a pre-determined volume of diluent to the primary chamber in a pre-determined time period when the diluent inlet is connected to a diluent supply.

2. The apparatus according to claim 1, wherein the flow controller comprises a flow restrictor configured to allow diluent to flow into the primary chamber at a pre-determined inlet flow rate, provided the diluent supply has an unrestricted flow rate greater than the pre-determined inlet flow rate.

3. The apparatus according to claim 2, wherein the pre-determined inlet flow rate is between 2.5 and 10 litres per minute.

4. The apparatus according to claim 1, comprising a receiving region for receiving a capsule having first and second cavities for storing the respective first and second parts, and an actuator operable to cause dispensing of the first and second parts from the capsule into the reagent inlet.

5. The apparatus according to claim 4, wherein the reagent inlet comprises a reaction chamber for receiving the first and second parts, the reaction chamber having an outlet for delivering a reagent mixture formed from the first and second parts to the primary chamber.

6. The apparatus according to claim 5, wherein the outlet comprises a restriction orifice for releasing the reagent mixture into the primary chamber at a controlled rate.

7. The apparatus according to claim 1, wherein the flow controller comprises a fill valve operable to stop and start the flow of diluent into the primary chamber.

8. (canceled)

9. An apparatus for preparing a liquid preparation using a two-part system comprising a first part including a first reagent and a second part including a second reagent, wherein the first reagent and the second reagent react when mixed to form an active ingredient of the liquid preparation, and wherein the liquid preparation comprises a diluent;
the apparatus comprising:
a primary chamber for receiving a quantity of the first part, a quantity of the second part and a quantity of the diluent;
a reagent inlet for admitting the first part and the second part to the primary chamber;
a diluent inlet connectable to a diluent supply for admitting the diluent to the primary chamber;
an outlet for releasing the liquid preparation from the primary chamber;
a fill valve operable to stop and start the flow of diluent into the primary chamber; and
a control module configured to:
receive a reagent delivery signal associated with the delivery of the first part and the second part to the primary chamber; and
cause operation of the fill valve in response to the reagent delivery signal.

10. The apparatus according to claim 9, wherein the control module is configured to provide a ready indication to the user after admission of the pre-determined quantity of diluent to the primary chamber to indicate that the preparation is ready for use.

11. The apparatus according to claim 10, wherein the control module is configured to wait for a pre-determined dwell period after admission of the pre-determined quantity of diluent to the primary chamber before providing the ready indication.

12. The apparatus according to claim 9, wherein the control module is configured to:
receive a start input signal before delivery of the first part and the second part to the primary chamber; and
cause operation of the fill valve in response to the start input signal to deliver a pre-delivery charge of the diluent to the primary chamber;
and wherein operation of the fill valve in response to the reagent delivery signal causes delivery of a post-delivery charge of the diluent to the primary chamber, the pre-delivery charge and post-delivery charge together having a total volume equal to said pre-determined quantity of diluent.

13. The apparatus according to claim 12, wherein the control module is configured to provide an activation indication to the user after delivery of the pre-delivery charge to indicate that the first part and the second part can be added to the primary chamber.

14. The apparatus according to claim 9, wherein operation of the fill valve in response to the reagent delivery signal admits said pre-determined volume of diluent to the primary chamber.

15. The apparatus according to claim 9, comprising a user input device operable by a user to cause the user interface to provide the reagent delivery signal to the control module.

16. (canceled)

17. The apparatus according to claim 9, comprising a delivery sensor configured to sense an event associated with the delivery of the first part and the second part to the primary chamber and to provide the reagent delivery signal to the control module upon sensing of the event.

18. (canceled)

19. The apparatus according to claim 9, comprising a diluent inlet flow sensor, the control module being configured to receive an inlet flow signal from the inlet flow sensor and to determine a diluent inlet flow rate based on the inlet flow signal.

20. (canceled)

21. (canceled)

22. The apparatus according to claim 9, comprising a fault sensor for detecting the presence of liquid in the primary chamber, the control module being configured to receive a fault signal from the fault sensor and to provide a fault indication to the user if the presence of liquid in the primary chamber is detected before operation of the fill valve.

23. (canceled)

24. The apparatus according to claim 9, comprising a data reader for reading data from a data carrier on a package containing the first part and/or the second part, the data relating to properties of the first part and/or the second part, the control module being configured to, prior to operation of the fill valve:
receive the data from the data reader;
validate the data against one or more pre-determined criteria; and
if the data does not satisfy the one or more pre-determined criteria, provide an error indication to the user.

25. The apparatus according to claim 9, comprising a drain port for emptying the primary chamber, the drain port having a drain valve operable by the control module.

26. (canceled)

27. (canceled)

28. The apparatus according to claim 9, comprising an outlet valve operable by the control module to control flow through the outlet.

29. (canceled)
